(19) United States
(12) Patent Application Publication
(10) Pub. No.: US 2019/0279776 A1
(43) Pub. Date: Sep. 12, 2019

(54) SYSTEMS AND METHODS USEFUL FOR PROVIDING AT-HOME VETERINARY CARE SERVICES

(71) Applicants: Alejandro Soriano, Washington, DC (US); Adrian Soriano, Washington, DC (US)

(72) Inventors: Alejandro Soriano, Washington, DC (US); Adrian Soriano, Washington, DC (US)

(21) Appl. No.: 16/297,626

(22) Filed: Mar. 9, 2019

Related U.S. Application Data
(60) Provisional application No. 62/641,331, filed on Mar. 10, 2018.

(51) Int. Cl. G16H 80/00; G16H 50/20; G06Q 10/08; H04L 12/58

(52) U.S. Cl. CPC G16H 80/00; H04L 51/046; G06Q 10/0875; G16H 50/20

(57) ABSTRACT

A system useful for facilitating veterinary care delivery includes a central server configured to communicate with a user device; a veterinary care service module stored on the central server; a processor configured to use the veterinary care service module to receive and process an electronic user request for veterinary services at a geographic location associated a user of the user device, the electronic user request communicated from the user device; and a veterinary care service provider database comprising veterinary care service provider data, wherein the processor uses the veterinary care service module to select and assign a veterinary care service provider based on the veterinary service provider data and the electronic user request for veterinary services, to communicate the selection to the user device, and to notify the selected veterinary care service provider of the selection.

### RELATED APPLICATIONS

This application claims the benefit of U.S. Provisional Application No. 62/641,331, titled AT HOME VETERINARY CARE SYSTEM, the entire disclosure of which is hereby incorporated herein by reference in its entirety.

### FIELD

The disclosure relates to systems and methods useful for providing veterinary care services. In particular, the disclosure relates to systems and methods for providing at-home veterinary care services.

### BACKGROUND

There is a recognized demand for veterinary care services that provide mobile care. Related art veterinary care services that provide mobile services outside of a brick and mortar office typically use a truck or van that serves as a clinic on wheels. Most mobile veterinary services are provided as an extension of brick and mortar office services, and offer limited convenience, but often at extra costs and with substantial limitations on the manner and extent of service provided.

### SUMMARY

A need has been recognized for enhanced veterinary care services including on-demand services by certified professionals at locations specified by pet owners to enhance pet safety, privacy, accessibility, and cost savings for providers and consuming user-clients. A need has been recognized for systems and associated processes that enable flexible work schedules and reduced overhead costs for veterinary care service providers. A need has been recognized for enhancing efficacy and efficiency of at home delivery of veterinary care services, diagnostic tests, and prescription and non-prescription treatments.

Systems and methods useful for providing at-home veterinary care services are disclosed that address, inter alia, the foregoing needs. In particular, systems and methods useful for providing veterinary care services to a pet at a location and time specified by the pet owner outside of a veterinary office are disclosed.

In some embodiments, the veterinary care system includes a memory that stores a set of instructions, and a processor or plurality of processors that use the set of instructions to process a veterinary care service request from a user. The stored instructions include instructions configured in accordance with methods and algorithms that select a doctor, a technician, or a combination thereof in response to the user request based at least in part in the type of visit specified by the user and the distance between the user and the selected care provider(s).

In some embodiments, the system uses geolocation systems and processes, now known and publicly available, or later developed, to identify where the user and the potentially assignable veterinary professionals or doctors are located. In some embodiments, the system allows users to schedule a veterinary visit request specifying a later date and time to receive the visit. In some embodiments, the system recommends to the doctor what medications and supplies to carry to the visit in a kit, doctor's bag or similar movable container based on, for example, the collection and analysis of historical data, geographical location, time of the year, past treatments, or the frequency with which veterinary patients experience certain types of health issues, among other factors.

In some embodiments, systems and methods enable the doctor using a smartphone, computer or similar device, to offer products as a result of diagnosing the patient (such as medicines, clinical tests or medicated foods, among others) and services (treatments) to the user-client and the client can review in real-time and approve those offers using another smartphone or similar device, thus providing the required consent to those products or services. In some embodiments, after approval by the client, the information may be sent over computer networks to automatically generate an electronic prescription, legally valid to be used to fill out additional veterinary products and services on the basis of an initial Veterinary Client Patient Relationship (VCPR).

In some embodiments, the system uses stored information to generate recommendations to the doctors about the most suitable products or services they can offer to their clients. In some embodiments, the system allows users and doctors to communicate after a house call visit using one or two-way video or text chat services, in accordance with telemedicine rules and practices valid in each jurisdiction after an initial VCPR is established. In some embodiments, the system uses stored information such as prescribed products and treatments performed to send electronic reminders to users regarding potential products or services, and to enhance compliance with veterinary medical treatments. In some embodiments, the system allows both doctors and users to provide feedback about their experience for promoting quality, safety, and other desirable benefits and advantages of at-home veterinary care.

In some embodiments, the system stores historical data about the patient from previous uses of this system, and from third party sources such lab results, records of visits to other veterinarians, and county and state records. In some embodiments, the system includes the deployment of one or more data collection devices, which may include wearable devices, mobile devices, and stationary devices placed at the user's home or in or on their pets in order to monitor health.

In some embodiments, the system enables doctors to summon additional moving services to the user's location to provide a more complete, at home solution to more complex or atypical veterinary issues, such as, for example, mobile imaging, among others. In some embodiments, the kit, the doctor's bag or equivalent movable container is designed to target the most common veterinary issues in a way that using predictive algorithms and historical frequencies the volume and effectiveness of the bag for providing at-home veterinary solutions can be optimized. In some embodiments, a central veterinary support system can enhance in real-time the ability of doctors to deliver more encompassing and effective at-home veterinary solutions by providing them with advice and current, reliable information.

In an embodiment, systems useful for facilitating veterinary care delivery include a central server configured to communicate with a user device; a veterinary care service module stored on the central server; a processor configured to use the veterinary care service module to receive and process an electronic user request for veterinary services at a geographic location associated a user of the user device, the electronic user request communicated from the user device; and a veterinary care service provider database comprising veterinary care service provider data, wherein the processor uses the veterinary care service module to select and assign a veterinary care service provider based on the veterinary service provider data and the electronic user request for veterinary services, to communicate the selection to the user device, and to notify the selected veterinary care service provider of the selection.

In an embodiment, systems include a treatment database, wherein the processor is configured to use the veterinary care service module to select a treatment option from the treatment database or veterinary care service provider identified treatment, and communicating the selected treatment option to the user device; receiving at the central server user selection or user approval electronically communicated from the user; and processing the user selection or approval to determine a treatment selection to be administered by the veterinary care provider at the geographic location associated with the user.

In an embodiment, systems include a test database, wherein the processor is configured to use the veterinary care service module to select a test option from the test database or a veterinary care service provider identified treatment, and communicating the selected test option to the user device; receiving at the central server user selection or user approval electronically communicated from the user; and processing the user selection or approval to determine a test selection to be administered by the veterinary care provider at the geographic location associated with the user.

In an embodiment, systems are configured to determine an estimated time of arrival based on a time specified by the user or based on the geographic location of the user and a geographic location associated with the selected veterinary care service provider.

In an embodiment, systems include the processor configured to use the veterinary care service module to process the electronic user request to determine a request for a specified appointment time for the veterinary services.

In an embodiment, systems include the processor configured to use the veterinary care service module to process the electronic user request to determine a request for a request for immediate veterinary services.

In an embodiment, systems include a veterinary care kit content module, the processor configured to use the veterinary care kit content module to determine items to include in the veterinary care kit to be delivered to the geographic location of the user or specified by the user. In an embodiment, the veterinary care kit content module is configured to determine the content based on at least one of the groups consisting of: pet patient history data, geographic location, time of year, past treatments, and non-patient specific health data.

In an embodiment, the central server is configured to communicate with a veterinary care service provider device. In an embodiment, the central server is configured to communicate with a second veterinary care service provider device.

In an embodiment, the processor is configured to use the veterinary care service module to select a treatment option from a treatment database or veterinary care service provider identified treatment, and communicate the selected treatment option to the user device.

In an embodiment, a treatment or test approval module is configured to generate an electronic prescription based on the approval for communication to the user device.

In an embodiment, systems include a patient history database configured to store pet treatment information, test information, and other patient history, the central server configured to enable access to the patient history database by the veterinary care service provider, the user, data collection devices, information from third party service providers, or a combination thereof.

In an embodiment, systems include a veterinary care communication module configured to enable one-way, two-way, or text chat communication between the veterinary care service provider and the user.

In an embodiment, systems include the processor configured to use the veterinary care service module to select and assign a second veterinary care service provider based on the veterinary service provider data, and wherein the selection and assignment of the first and second veterinary care service providers are based on geolocation data associated with the first and second veterinary care service providers and the user or user device.

In an embodiment, methods useful for facilitating veterinary care delivery include providing a user input portal configured for communicating between a mobile device and a central server; receiving, at the central server an electronically communicated service order for veterinary services; processing, using a processor associated with the central server, the electronically communicated service order at the central server to determine information comprising patient history, service, location, service time, or a combination thereof determining, based on the processing, whether the service time requested is immediate; communicating an electronic confirmation of the requested appointment time, or an estimated time of arrival, or a combination thereof and automatically selecting a veterinary care service provider, using a veterinary care service provider database, a veterinary care service provider module, and a processor associated with the central server.

In another embodiment, methods include automatically selecting, using the processor associated with the central server, the veterinary care provider database, and the veterinary care service module, a veterinary care service provider, at least a second veterinary care service provider, or a combination thereof, from a plurality of veterinary care service providers based on the service order information, pet patient information, location data associated with the user or pet, veterinary care service provider information, or a combination thereof.

In another embodiment, methods include automatically selecting, sorting, or a combination thereof, using a processor associated with the central server, the veterinary care treatment module, and a treatment database, a prescription or non-prescription treatment based on veterinary care service provider input, user, input, and/or pet patient information.

In another embodiment, methods include automatically selecting, sorting, or a combination thereof, using a processor associated with the central server, a veterinary care test module, and a test database, a diagnostic test based on veterinary care service provider input, test information, user provided information, and/or pet patient information.

In another embodiment, methods include automatically selecting, sorting, or a combination thereof, using a processor associated with the central server, a veterinary care kit module, and a kit database, a veterinary care kit based on veterinary care service provider input, kit information, user provided information, and/or pet patient information.

In another embodiment, methods include communicating selections to a user device from central server for acceptance and/or confirmation by user and/or for display through user input portal.

Additional features and technical effects of the present disclosure will become readily apparent to those skilled in the art from the following detailed description wherein embodiments of the present disclosure are described simply by way of illustration of the best mode contemplated to carry out the present disclosure. In addition to the embodiments disclosed herein, other and different embodiments are within the spirit and scope of the disclosure, and its several details are capable of modifications in various respects.

### BRIEF DESCRIPTION OF DRAWINGS

The disclosure is shown by way of example in the figures of the accompanying drawings, in which like reference numerals refer to like elements.

FIG. 1 shows a diagrammatical plan view of systems useful for providing veterinary care services in accordance with an exemplary embodiment;

FIG. 2 shows methods useful for providing veterinary care services in accordance with an exemplary embodiment;

FIG. 3 shows methods useful for providing veterinary care services in accordance with an exemplary embodiment;

FIG. 4 shows methods useful for providing veterinary care services in accordance with an exemplary embodiment;

FIG. 5 shows a user portal displayed on a user device communicating with veterinary care systems and methods in accordance with an exemplary embodiment;

FIG. 6 shows a user portal displayed on a user device communicating with veterinary care systems and methods in accordance with an exemplary embodiment;

FIG. 7 shows a user portal displayed on a user device communicating with veterinary care systems and methods in accordance with an exemplary embodiment;

FIG. 8 shows a user portal displayed on a user device communicating with veterinary care systems and methods in accordance with an exemplary embodiment;

FIG. 9 shows a user portal displayed on a user device communicating with veterinary care systems and methods in accordance with an exemplary embodiment;

FIG. 10 shows a user portal displayed on a user device communicating with veterinary care systems and methods in accordance with an exemplary embodiment;

FIG. 11 shows a user portal displayed on a user device communicating with veterinary care systems and methods in accordance with an exemplary embodiment;

FIG. 12 shows a user portal displayed on a user device communicating with veterinary care systems and methods in accordance with an exemplary embodiment.

### DETAILED DESCRIPTION OF EMBODIMENTS

In the following description, for the purposes of explanation, numerous specific details are set forth in order to provide a thorough understanding of exemplary embodiments. It should be apparent, however, that exemplary embodiments may be practiced without these specific details or with an equivalent arrangement. In other instances, well-known structures and devices are shown in block diagram form in order to avoid unnecessarily obscuring exemplary embodiments. In addition, unless otherwise indicated, all numbers expressing quantities, ratios, and numerical properties of ingredients, reaction conditions, and so forth used in the specification and claims are to be understood as being modified in all instances by the term “about.”

Systems and methods useful for providing at home veterinary care services are disclosed. In particular, systems and methods useful for providing veterinary care services to a pet at a location and time specified by the pet owner outside of a veterinary office are disclosed. The disclosed systems and processes enhance pet safety, privacy, accessibility, and cost savings for providers and consuming user-clients. The disclosed systems and processes enable flexible work schedules and reduce overhead costs for veterinary care service providers. A need has been recognized for enhancing efficacy and efficiency of at home delivery of veterinary care services, diagnostic tests, and prescription and non-prescription treatments and the disclosed systems and methods provide a solution.

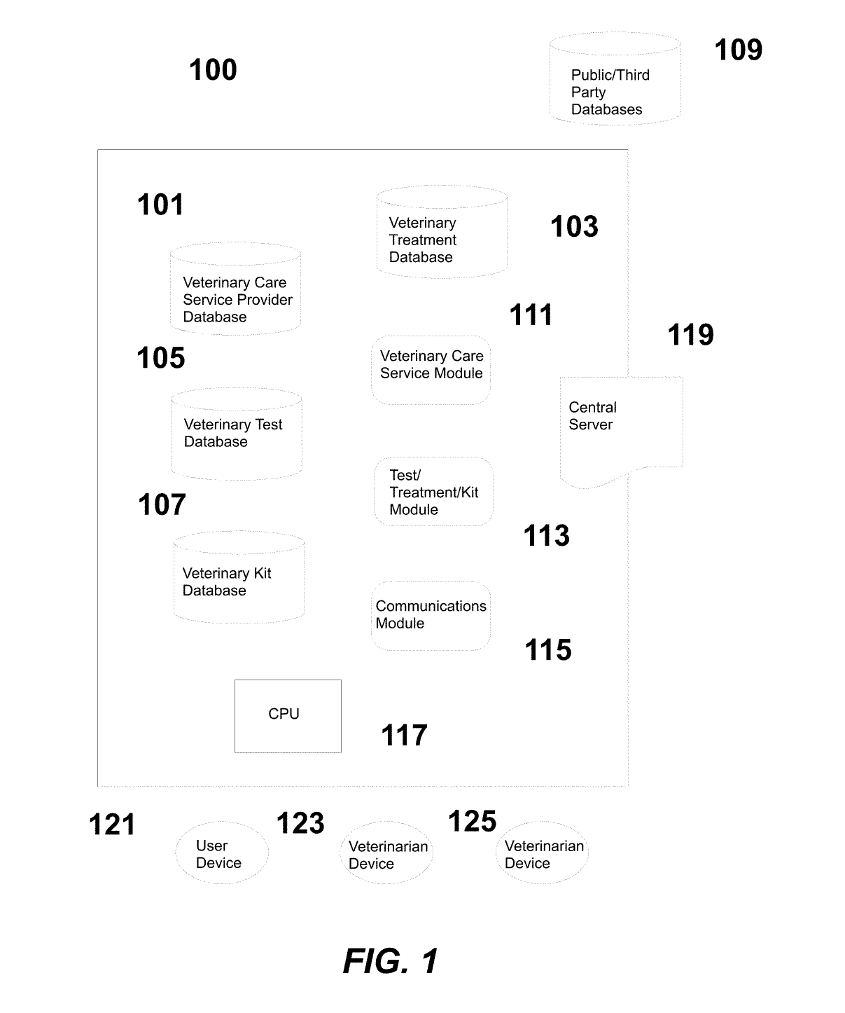

FIG. 1 shows a diagrammatical plan view of systems useful for providing veterinary care services in accordance with an exemplary embodiment. In particular, FIG. 1 shows a system 100 including a veterinary service provider database 101 in association with an on-demand veterinary care service provider system 103. The system 103 may be associated with a veterinary test database 105, a veterinary kit database 107, an external database 109, or a combination thereof, as shown in FIG. 1.

The system 101 includes computer readable instructions configured to facilitate processing of user-client service requests based on patient data, historical data, third party data, geolocation data of clients, pets, service providers, or a combination thereof. The instructions may be configured to facilitate selection of care providers, communication with clients, selection of tests, treatments, and doctor and client communication by text and video chat. For example, the system 101 may include a veterinary care service module 111, a test and/or treatment module 113, and a communications module 115, or a combination thereof as shown in FIG. 1.

The system 101 is associated with a computer processing unit 117, or a plurality of processors. The processor 117 and one or more of the modules 111, 113, and 115, and one or more of databases, 101, 105, and 107, may be configured to automatically process electronically communicated user service requests, match service providers, compile kit stock lists, offer tests, and treatments, and facilitate prescription delivery.

The system 101 may be associated with a central server 119 configured to communicate with external databases, modules, and systems, and devices of users and care providers. The central server 119 may be configured to facilitate communication between the system 101 and a user device 121, a veterinarian device 123, and a veterinarian device 125. The veterinary devices 123 and 125 may be a device of a licensed veterinarian or a veterinary care technician. The communications module 115 may be configured to facilitate communication by text or video between a user-client and one or more veterinary care providers.

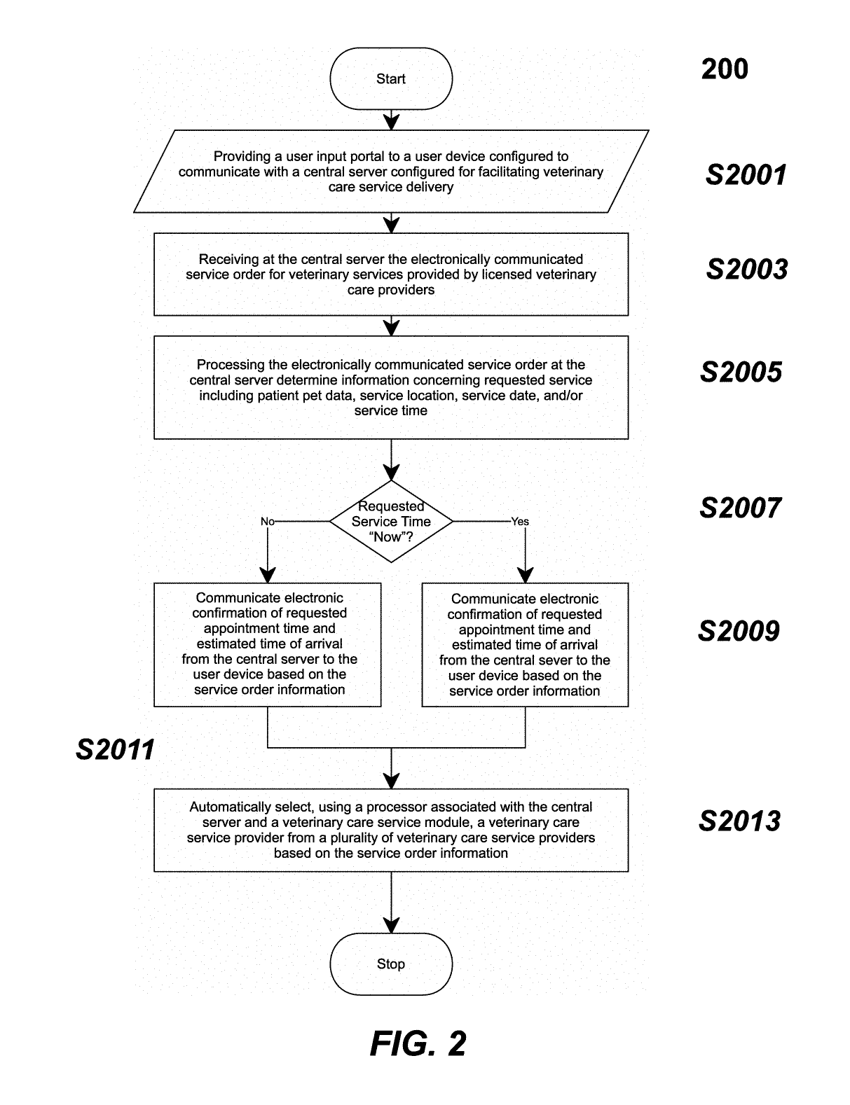

FIG. 2 shows methods useful for providing veterinary care services in accordance with an exemplary embodiment. In particular, FIG. 2 shows a process 200 including a first step S2001 of providing a user input portal to a user device configured to communicate with a central server configured for facilitating veterinary care service delivery. The process 200 includes, at S2003, receiving at the central server the electronically communicated service order for veterinary services provided by veterinary care providers. Subsequently, at S2005, the process 200 includes processing the electronically communicated service order at the central server to determine information concerning the requested service including patient pet data, service location, service date, service time, or a combination thereof. The system may determine, at S2007, that the service request includes a request for immediate or present services. If so, the process 200 includes communicating, at S2009, an electronic confirmation of a requested appointment time and an estimated time of arrival from the central server to the user device based on the service order information.

If the system determines, at S2007, that the service request includes a request for a scheduled time, the process 200 includes, at S2011 communicating electronic confirmation of a requested appointment time from the central server to a user device based on the service order information.

The process 200 shown in FIG. 2 shows a step S2013 of automatically selecting, using a processor associated with the central server and a veterinary care service module, a veterinary care service provider from a plurality of veterinary care service providers based on the service order information.

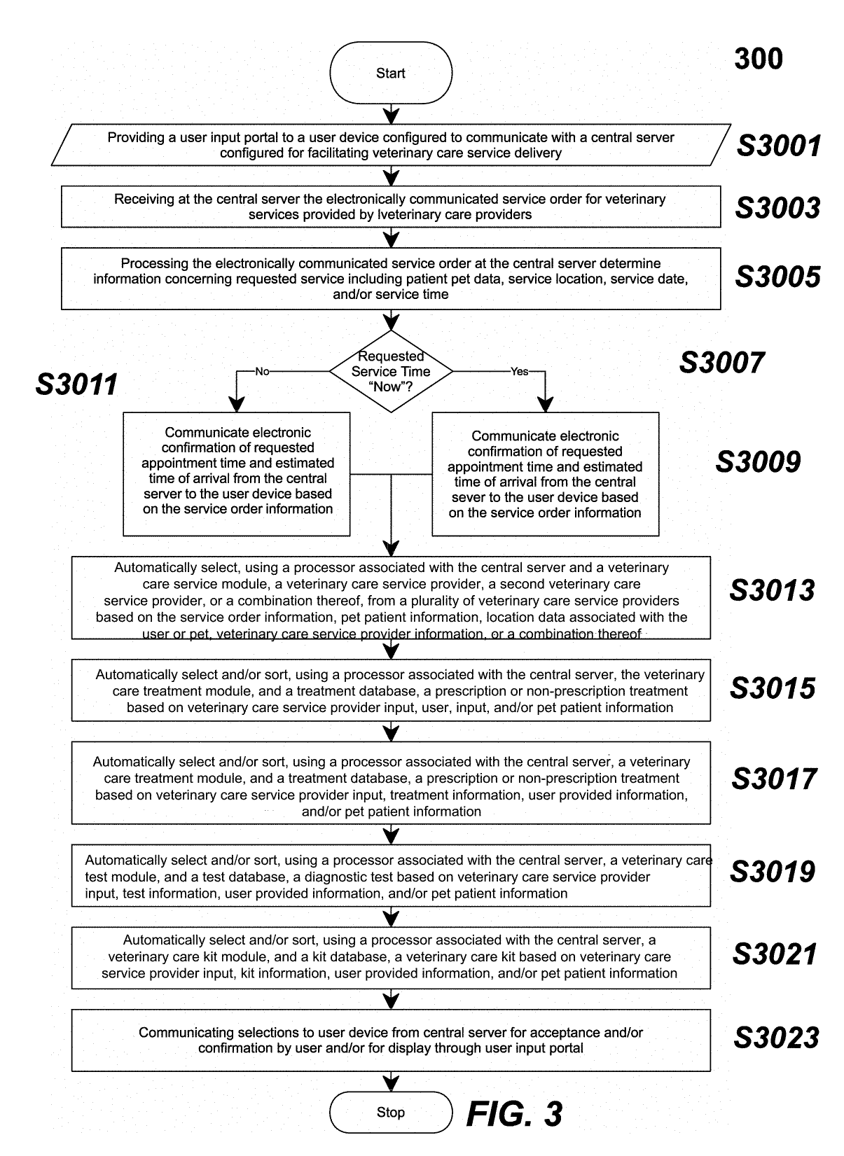

FIG. 3 shows methods useful for providing veterinary care services in accordance with an exemplary embodiment. In particular, FIG. 3 shows a process 300 including a first step S3001 of providing a user input portal to a user device configured to communicate with a central server configured for facilitating veterinary care service delivery. Subsequently, at S3003, the process 300 includes receiving at the central server the electronically communicated service order for veterinary services provided by veterinary care providers. The process 300 includes processing, at S3005, the electronically communicated service order at the central server determine information concerning requested service including patient pet data, service location, service date, service time, or a combination thereof.

The system may be configured to determine, at S3007, that the service request includes a request for immediate or present services. If so, the process 300 includes communicating, at S3009, an electronic confirmation of a requested appointment time and an estimated time of arrival from the central server to the user device based on the service order information.

If the system determines, at S3007, that the service request includes a request for a scheduled time, the process 300 includes, at S3011 communicating electronic confirmation of a requested appointment time from the central server to a user device based on the service order information.

In an embodiment, processes may include a step S3013 of automatically selecting, using a processor associated with the central server and a veterinary care service module, a veterinary care service provider, at least a second veterinary care service provider, or a combination thereof, from a plurality of veterinary care service providers based on the service order information, pet patient information, location data associated with the user or pet, veterinary care service provider information, or a combination thereof. As shown in FIG. 3, processes may include automatically selecting, sorting, or a combination thereof, using a processor associated with the central server, the veterinary care treatment module, and a treatment database, a prescription or non-prescription treatment based on veterinary care service provider input, user, input, and/or pet patient information, at S3015.

The process in FIG. 3 shows a step S3017 of automatically selecting, sorting, or a combination thereof, using a processor associated with the central server, a veterinary care treatment module, and a treatment database, a prescription or non-prescription treatment based on veterinary care service provider input, treatment information, user provided information, pet patient information, or a combination thereof. Processes of embodiments may include a step S3019 of automatically selecting, using a processor associated with the central server, a veterinary care test module, and a test database, a diagnostic test based on veterinary care service provider input, test information, user provided information, and/or pet patient information. Further, processes of embodiments may include a step S3021 of automatically selecting, using a processor associated with the central server, a veterinary care kit module, and a kit database, a veterinary care kit based on veterinary care service provider input, kit information, user provided information, and/or pet patient information.

Processes may include a step S3023 of communicating selections or sorting to a user device from the central server for acceptance and/or confirmation by a user and/or for display through the user input portal.

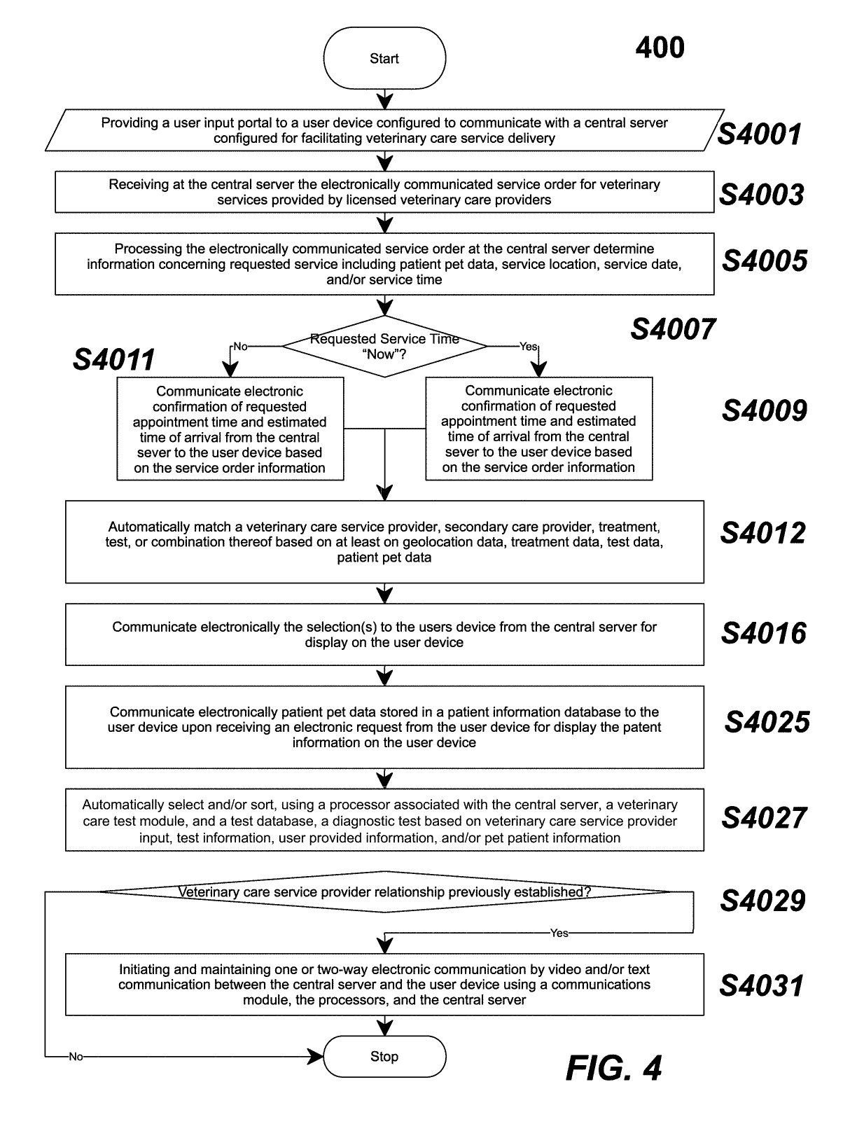

FIG. 4 shows methods useful for providing veterinary care services in accordance with an exemplary embodiment. In particular, FIG. 4 shows a process 400 including a first step S4001 of providing a user input portal to a user device configured to communicate with a central server configured to facilitate veterinary care service delivery. Subsequently, at S4003, the process 400 includes receiving at the central server the electronically communicated service order for veterinary services provided by licensed veterinary care providers. The process 400 includes processing, at S4005, the electronically communicated service order at the central server determine information concerning requested service including patient pet data, service location, service date, service time, or a combination thereof.

The system may determine, at S4007, that the service request includes a request for immediate or present services. If so, the process 400 includes communicating, at S4009 an electronic confirmation of a requested appointment time and an estimated time of arrival from the central sever to the user device based on the service order information.

If the system determines, at S4007, that the service request includes a request for a scheduled time, the process 400 includes, at S4011 communicating electronic confirmation of a requested appointment time from central server to user device based on the service order information.

In an embodiment, processes may include a step S4012 of automatically matching a veterinary care service provider, secondary care provider, treatment, test, or combination thereof based on at least on geolocation data, treatment data, test data, patient pet data, or a combination thereof. In an embodiment, processes may include, as shown in FIG. 4, a step S4016 of communicating electronically the selection(s) to the users device from the central server for display on the user device. The process 400 includes a step S4025 of communicating electronically patient pet data stored in a patient information database to the user device upon receiving an electronic request from the user device for display the patent information on the user device. The process 400 includes a step S4027 of automatically selecting, using a processor associated with the central server, a veterinary care test module, and a test database, a diagnostic test based on veterinary care service provider input, test information, user provided information, pet patient information, or a combination thereof.

In an embodiment, at S4029, the process 400 includes a step of determining whether a veterinary care service provider relationship has been established. The relationship may be one that meets the requirements of a Veterinary Client Patient Relationship (VCPR). If the determination at S4031 is yes, then the process 400 includes a step S4031 of initiating and maintaining one or two-way electronic communication by video and/or text communication between the central server and the user device using a communications module, the processors, and the central server.

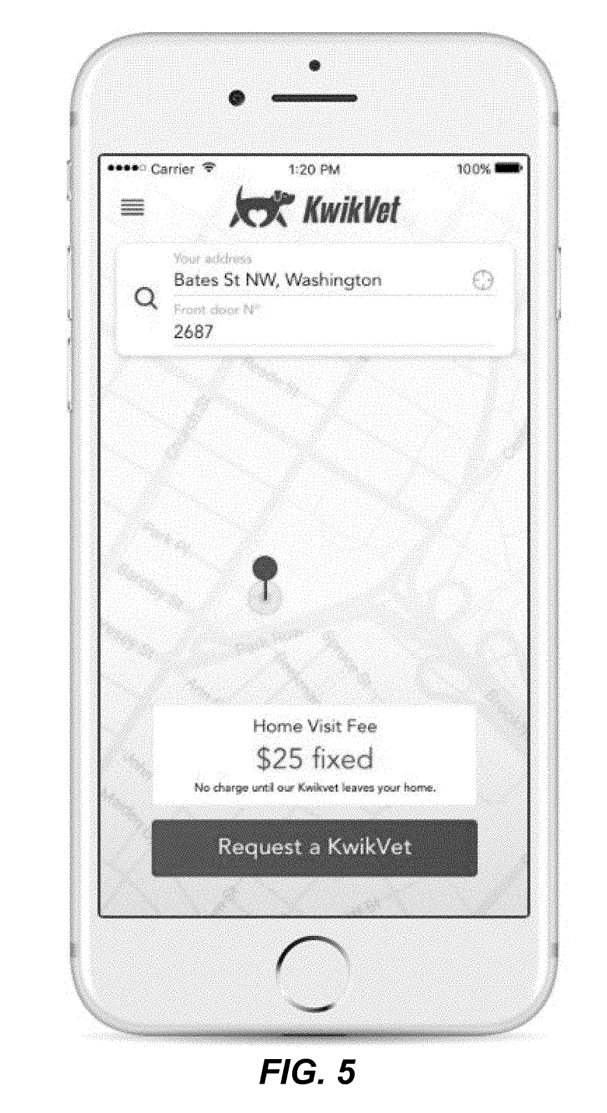

Processes may include communicating a user portal to a user device or veterinary care provider device. The portal may enable communication with the veterinary care service provider system. For example, FIG. 5 shows a user portal and associated ornamental design displayed on a user device communicating with veterinary care systems and methods in accordance with an exemplary embodiment. FIG. 5 shows input fields displayed on the user device for address information, visual location information, displayed fees and terms, and a request button for submitting a request for services.

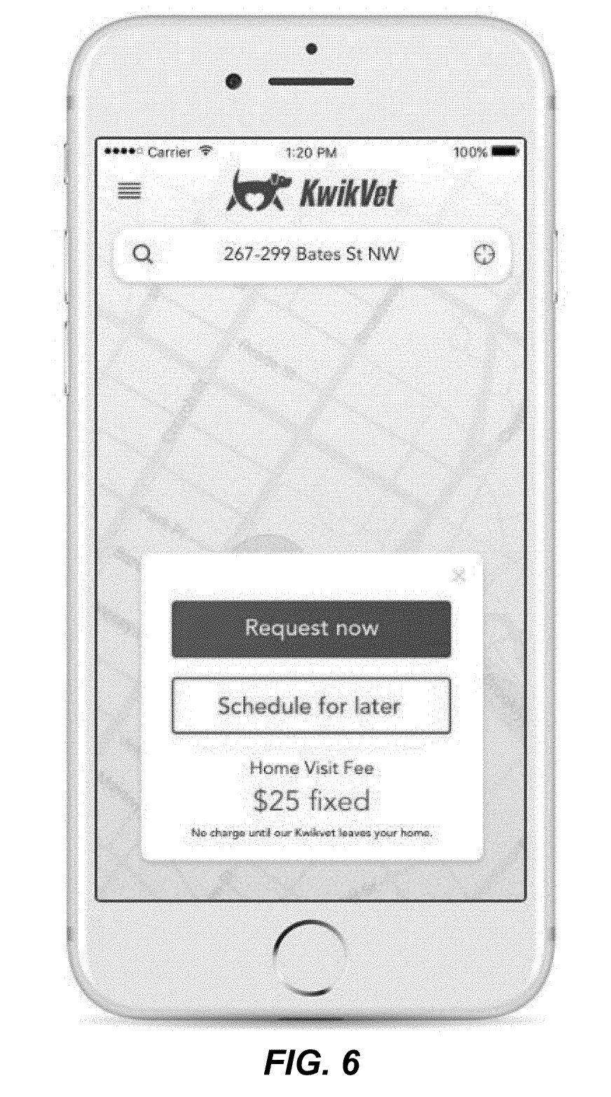

FIG. 6 shows a user portal displayed on a user device communicating with veterinary care systems and methods in accordance with an exemplary embodiment. For example, FIG. 6 shows a user portal and associated ornamental design displayed on a user device communicating with veterinary care systems and methods in accordance with an exemplary embodiment. FIG. 6 shows option input buttons for scheduling service, and displays fees and terms.

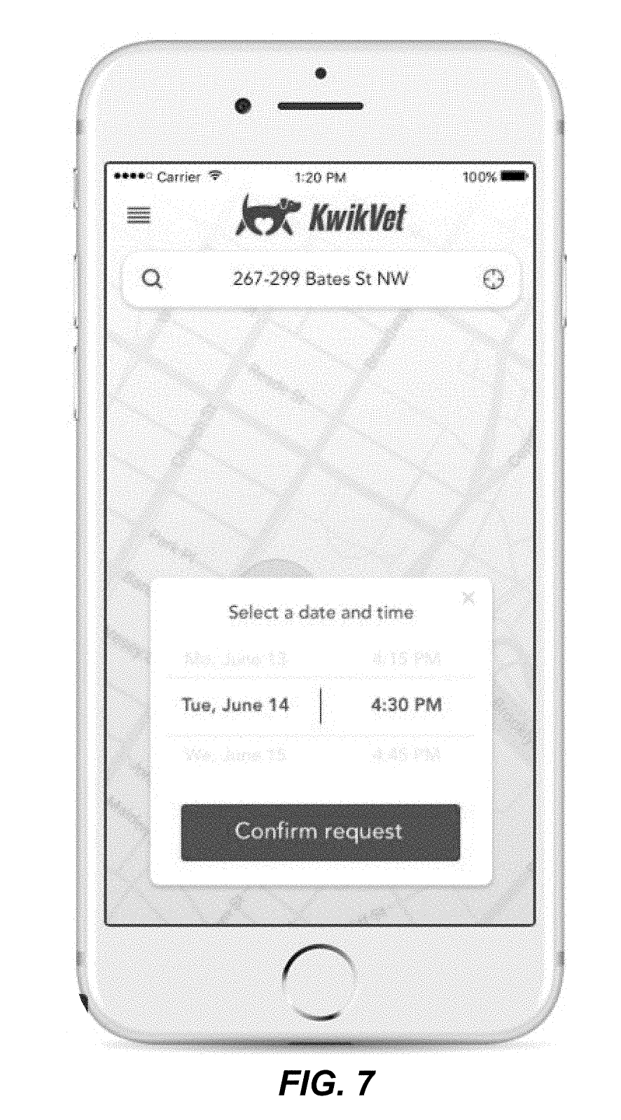

FIG. 7 shows a user portal displayed on a user device communicating with veterinary care systems and methods in accordance with an exemplary embodiment. For example, FIG. 7 shows a user portal and associated ornamental design displayed on a user device communicating with veterinary care systems and methods in accordance with an exemplary embodiment. FIG. 7 shows scheduling input fields, location information, and an option button for confirming service order request for submission.

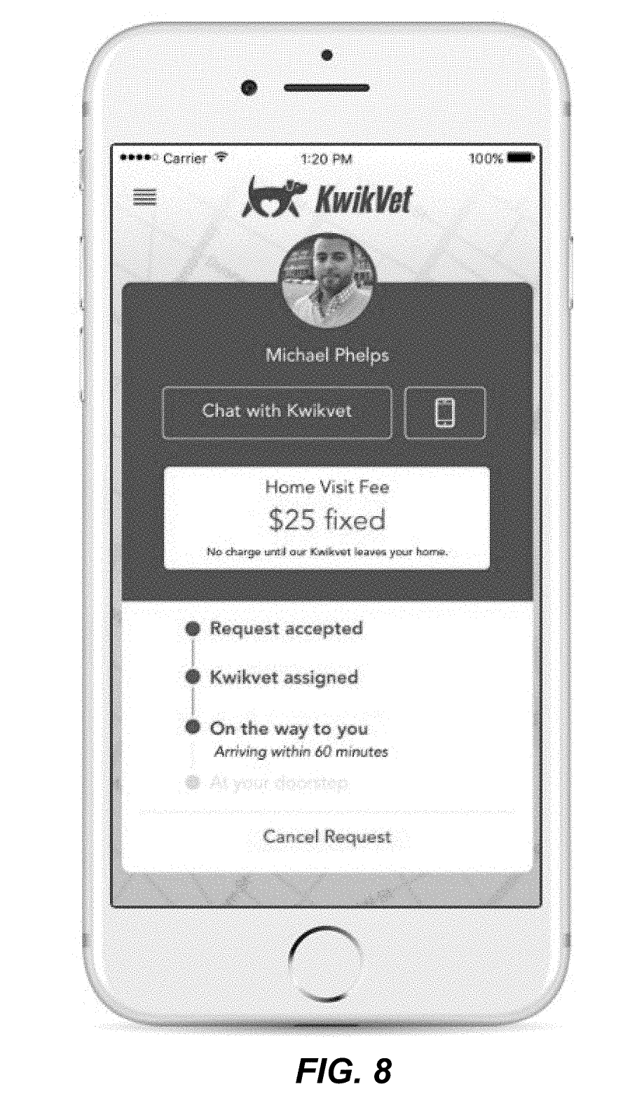

FIG. 8 shows a user portal displayed on a user device communicating with veterinary care systems and methods in accordance with an exemplary embodiment. For example, FIG. 8 shows a user portal and associated ornamental design displayed on a user device communicating with veterinary care systems and methods in accordance with an exemplary embodiment. FIG. 8 shows displayed fees and term, and indication of order status, veterinary care provider selection and assignment, indication of estimated time of arrival and location of care provider, and identity of care provider. FIG. 8 also shows a displayed button for canceling a service request from the user device.

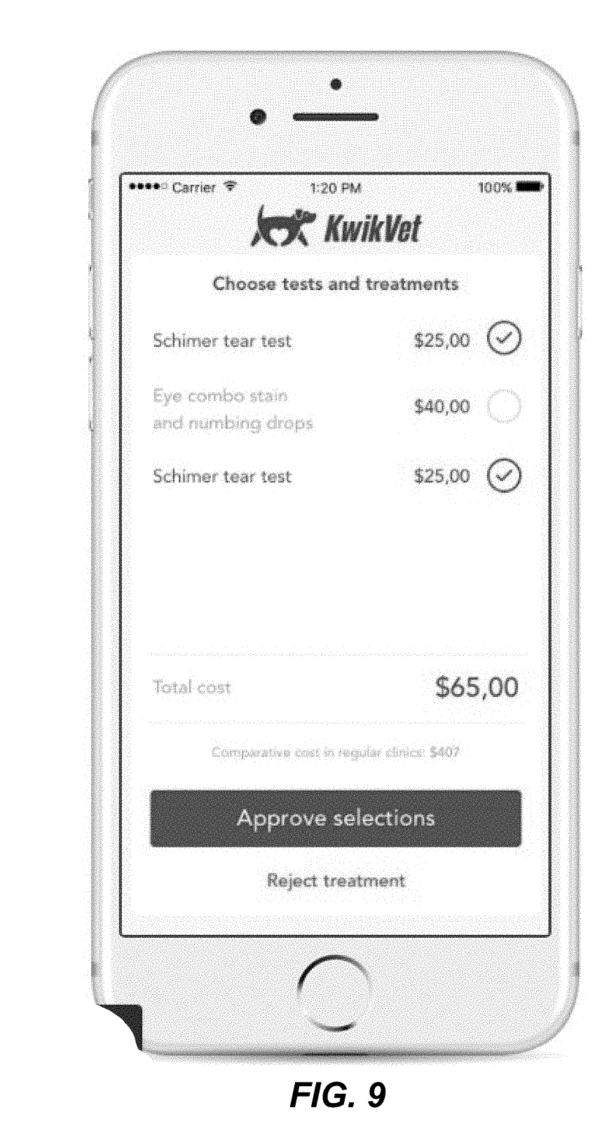

FIG. 9 shows a user portal displayed on a user device communicating with veterinary care systems and methods in accordance with an exemplary embodiment. For example, FIG. 9 shows a user portal and associated ornamental design displayed on a user device communicating with veterinary care systems and methods in accordance with an exemplary embodiment. FIG. 9 shows displayed test and treatment options, input fields for selection, displayed buttons for approval of selection and rejection of treatment, and prices and total cost with relative comparison pricing.

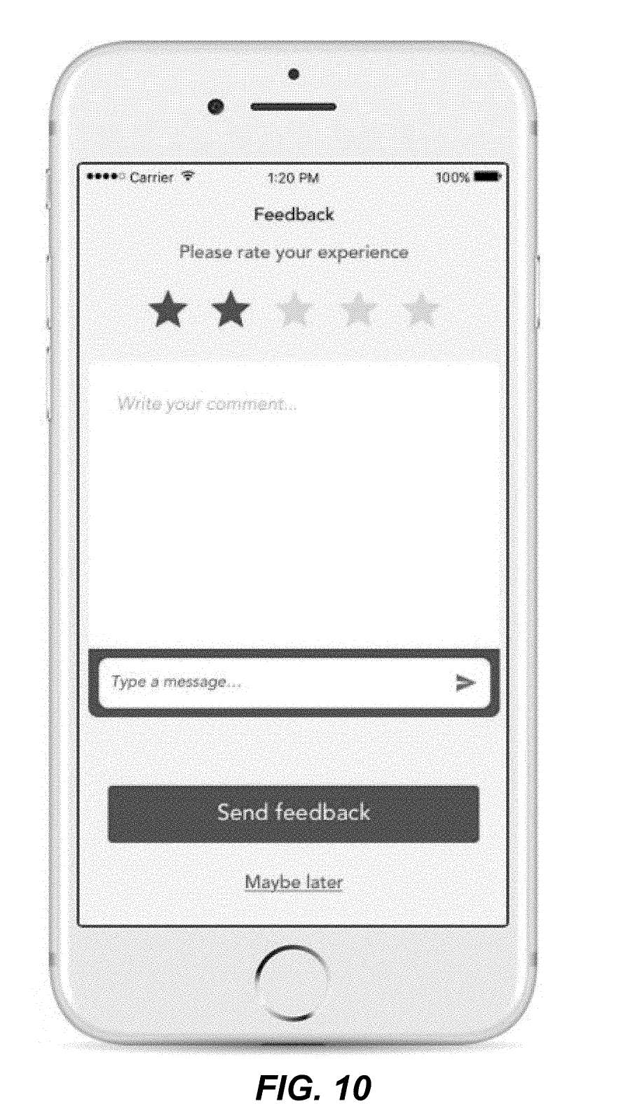

FIG. 10 shows a user portal displayed on a user device communicating with veterinary care systems and methods in accordance with an exemplary embodiment. For example, FIG. 10 shows a user portal and associated ornamental design displayed on a user device communicating with veterinary care systems and methods in accordance with an exemplary embodiment. FIG. 10 shows input fields displayed on the user device for communication with a veterinary care service provider system, a displayed input field for feedback and ratings fields, along with a displayed option button for sending feedback.

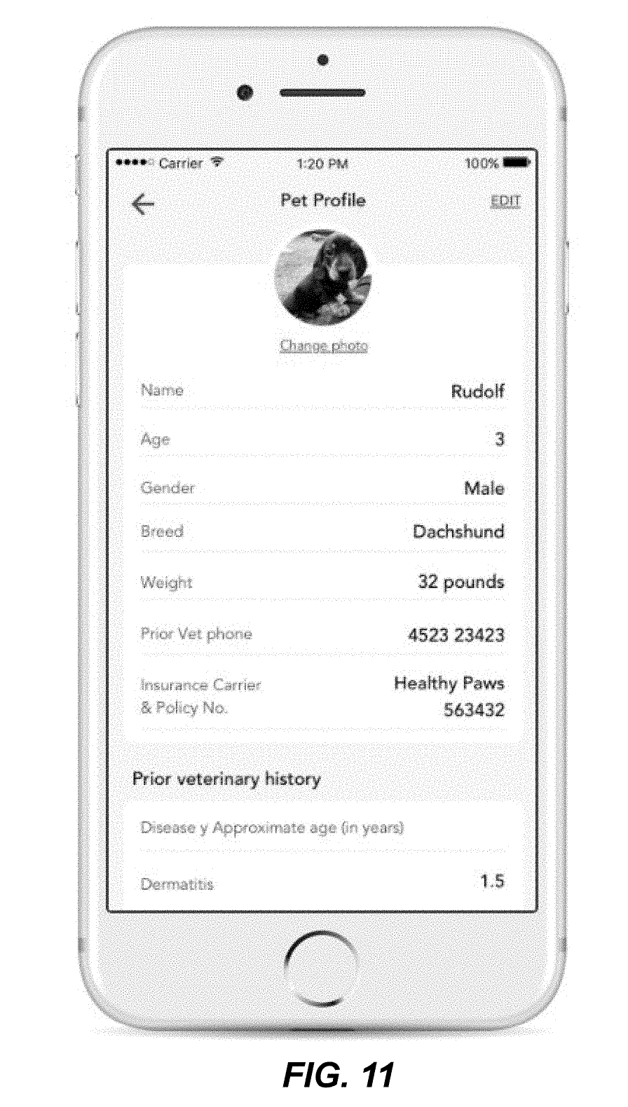

FIG. 11 shows a user portal displayed on a user device communicating with veterinary care systems and methods in accordance with an exemplary embodiment. For example, FIG. 11 shows a user portal and associated ornamental design displayed on a user device communicating with veterinary care systems and methods in accordance with an exemplary embodiment. FIG. 11 shows displayed pet patient data and identify information including patient history data.

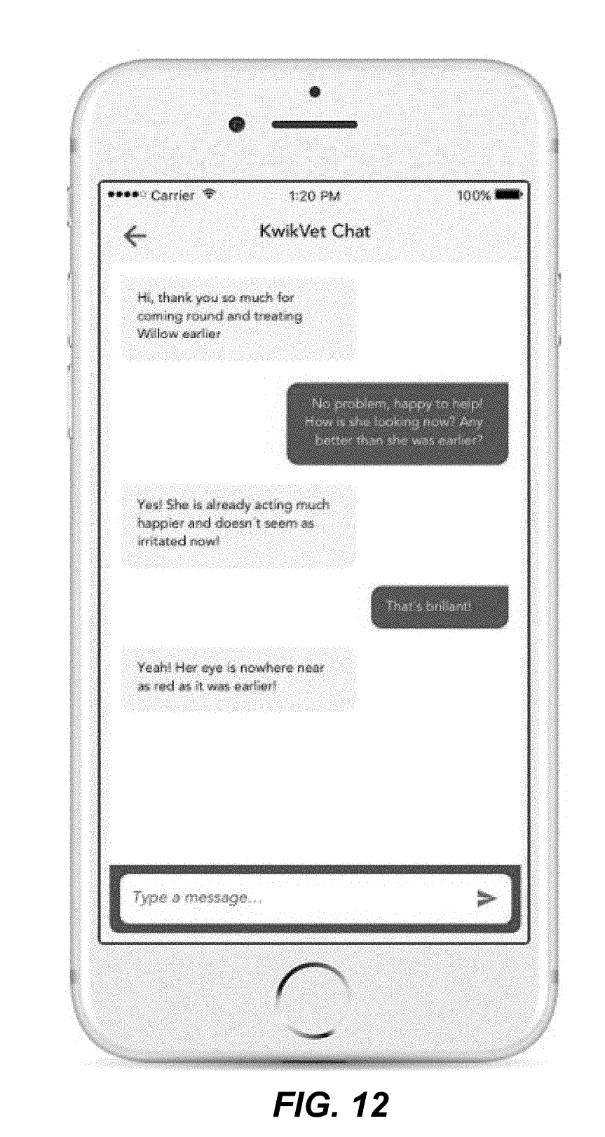

FIG. 12 shows a user portal displayed on a user device communicating with veterinary care systems and methods in accordance with an exemplary embodiment. For example, FIG. 12 shows a user portal and associated ornamental design displayed on a user device communicating with veterinary care systems and methods in accordance with an exemplary embodiment. FIG. 12 shows input fields displayed on the user device for messaging, and displayed text messages between care provider and user-client.

Embodiments are shown by way of example, and not by way of limitation in the figures and drawings. While the invention has been described in connection with a number of embodiments and implementations, the invention is not so limited but covers various obvious modifications and equivalent arrangements, which fall within the purview of the appended claims. Although features of the invention are expressed in certain combinations among the claims, it is contemplated that these features can be arranged in any combination and order.

What is claimed is:

1. A system useful for facilitating veterinary care delivery, comprising:
a central server configured to communicate with a user device;
a veterinary care service module stored on the central server;
a processor configured to use the veterinary care service module to receive and process an electronic user request for veterinary services at a geographic location associated with a user of the user device, the electronic user request communicated from the user device; and
a veterinary care service provider database comprising veterinary care service provider data, wherein the processor uses the veterinary care service module to select and assign a veterinary care service provider based on the veterinary service provider data and the electronic user request for veterinary services, to communicate the selection to the user device, and to notify the selected veterinary care service provider of the selection.

2. The system of claim 1, comprising:
a treatment database, wherein the processor is configured to use the veterinary care service module to select a treatment option from the treatment database or veterinary care service provider identified treatment, and communicating the selected treatment option to the user device;
receiving at the central server user selection or user approval electronically communicated from the user; and
processing the user selection or approval to determine a treatment selection to be administered by the veterinary care provider at the geographic location associated with the user.

3. The system of claim 1, comprising:
a test database, wherein the processor is configured to use the veterinary care service module to select a test option from the test database or a veterinary care service provider identified treatment, and communicating the selected test option to the user device;
receiving at the central server user selection or user approval electronically communicated from the user; and
processing the user selection or approval to determine a test selection to be administered by the veterinary care provider at the geographic location associated with the user.

4. The system of claim 1, comprising:
determining an estimated time of arrival based on a time specified by the user or based on the geographic location of the user and a geographic location associated with the selected veterinary care service provider.

5. The system of claim 1, comprising the processor configured to use the veterinary care service module to process the electronic user request to determine a request for a specified appointment time for the veterinary services.

6. The system of claim 1, comprising the processor configured to use the veterinary care service module to process the electronic user request to determine a request for a request for immediate veterinary services.

7. The system of claim 1, comprising:
a veterinary care kit content module, the processor configured to use the veterinary care kit content module to determine items to include in the veterinary care kit to be delivered to the geographic location of the user or specified by the user.

8. The system of claim 7, comprising the veterinary care kit content module configured to determine the items based on at least one of the groups consisting of: pet patient history data, geographic location, time of year, past treatments, and non-patient specific health data.

9. The system of claim 8, comprising the central server configured to communicate with a veterinary care service provider device.

10. The system of claim 9, comprising the central server configured to communicate with a second veterinary care service provider device.

11. The system of claim 10, the processor configured to use the veterinary care service module to select a treatment option from a treatment database or veterinary care service provider identified treatment, and communicating the selected treatment option to the user device.

12. The system of claim 2, comprising:
a treatment or test approval module configured to generate an electronic prescription based on the approval for communication to the user device.

13. The system of claim 1, comprising:
a patient history database configured to store pet treatment information, test information, and other patient history, the central server configured to enable access to the patient history database by the veterinary care service provider, the user, data collection devices, information from third party service providers, or a combination thereof.

14. The system of claim 1, comprising:
a veterinary care communication module configured to enable one-way, two-way, or text chat communication between the veterinary care service provider and the user.

15. The system of claim 1, wherein the processor uses the veterinary care service module to select and assign a second veterinary care service provider based on the veterinary service provider data, and wherein the selection and assignment of the first and second veterinary care service providers are based on geolocation data associated with the first and second veterinary care service providers and the user or user device.

16. A method useful for facilitating veterinary care delivery, comprising:
providing a user input portal configured for communicating between a mobile device and a central server;
receiving, at the central server an electronically communicated service order for veterinary services;
processing, using a processor associated with the central server, the electronically communicated service order at the central server to determine information comprising patient history, service, location, service time, or a combination thereof;
determining, based on the processing, whether the service time requested is immediate;
communicating an electronic confirmation of the requested appointment time, or an estimated time of arrival, or a combination thereof; and
automatically selecting a veterinary care service provider, using a veterinary care service provider database, a veterinary care service provider module, and a processor associated with the central server.

17. The method of claim 16, comprising:
automatically selecting, using the processor associated with the central server, the veterinary care provider database, and the veterinary care service module, a veterinary care service provider, at least a second veterinary care service provider, or a combination thereof, from a plurality of veterinary care service providers based on the service order information, pet patient information, location data associated with the user or pet, veterinary care service provider information, or a combination thereof.

18. The method of claim 16, comprising:
automatically selecting or sorting, using a processor associated with the central server, the veterinary care treatment module, and a treatment database, a prescription or non-prescription treatment based on veterinary care service provider input, user, input, and/or pet patient information.

19. The method of claim 16, comprising:
automatically selecting or sorting, using a processor associated with the central server, a veterinary care test module, and a test database, a diagnostic test based on veterinary care service provider input, test information, user provided information, and/or pet patient information;
automatically selecting or sorting, using a processor associated with the central server, a veterinary care kit module, and a kit database, a veterinary care kit based on veterinary care service provider input, kit information, user provided information, and/or pet patient information

20. The method of claim 16, comprising:
communicating selections to user device from central server for acceptance and/or confirmation by user and/or for display through user input portal.
